(19) United States
(12) Patent Application Publication
(10) Pub. No.: US 2011/0245655 A1
(43) Pub. Date: Oct. 6, 2011

(54) MAGNETIC RESONANCE IMAGING APPARATUS AND PULSE SEQUENCE ADJUSTING METHOD

(76) Inventors: Takayuki Abe, Tokyo (JP); Masahiro Takizawa, Tokyo (JP); Tetsuhiko Takahashi, Tokyo (JP)

(21) Appl. No.: 13/139,041

(22) PCT Filed: Dec. 22, 2009
(86) PCT No.: PCT/JP2009/071288

§ 371 Date: Jun. 10, 2011

(30) Foreign Application Priority Data
Dec. 26, 2008 (JP) 2008-333811
Jun. 9, 2009 (JP) 2009-146340

(51) Int. Cl. A61B 5/055

(52) U.S. Cl. 600/410

(57) ABSTRACT

When executing an imaging pulse sequence using a high frequency magnetic field pulse with a partial waveform of a predetermined waveform, an application start time of a slice gradient magnetic field applied simultaneously with the high frequency magnetic field pulse is corrected. Specifically, a magnetic resonance signal for correcting the imaging pulse sequence is acquired by executing a prescan sequence using a high frequency magnetic field pulse with a predetermined waveform, an application start time of a slice selection gradient magnetic field in the imaging pulse sequence is corrected using the magnetic resonance signal for correction, and the imaging pulse sequence is executed by applying the slice selection gradient magnetic field with the corrected application start time.

### TECHNICAL FIELD

The present invention relates to a magnetic resonance imaging apparatus (hereinafter, referred to as an MRI apparatus) and in particular, to an MRI apparatus, which performs slice-selective excitation using a half-wave high frequency pulse and performs UTE imaging for measuring a signal within an ultra-short echo time (UTE), and a pulse sequence adjusting method.

### BACKGROUND ART

In the MRI apparatus, when generating a nuclear magnetic resonance signal by exciting the nuclear spin of a subject, a slice selection gradient magnetic field is applied together with a high frequency magnetic field pulse in order to selectively excite a specific region. As the high frequency magnetic field pulse, a high frequency modulated by an envelope, such as a symmetric sinc function, is usually used. A profile obtained by the frequency-direction Fourier transform of the high frequency magnetic field modulated by the sinc function is a rectangle, and a predetermined rectangular region determined by the slice gradient magnetic field is excited.

Instead of the high frequency magnetic field pulse (this is called a full RF pulse) having the above-described symmetric function as an envelope (predetermined waveform), there is a method using a high frequency magnetic field pulse (called a half RF pulse) with a waveform of the half (partial waveform of a predetermined waveform) (PTLs 1 and 2, and the like). The half RF pulse is a pulse using only a waveform of the first half when dividing a symmetric sinc pulse into the front and the rear in a time direction with the peak in the middle, for example. By applying this method, a signal can be measured within a very short time (TE) from the spin excitation by measuring a signal from the rising time of a readout gradient magnetic field without using a phase encoding gradient magnetic field and without using a dephasing gradient magnetic field as a readout gradient magnetic field when measuring an echo. This imaging method is called ultra-short TE imaging (UTE imaging). Since the UTE imaging can shorten the TE further in this way, applications to imaging of the tissue with a short transverse relaxation time T2 which was difficult to be imaged with a conventional MRI, for example, the bone tissue and the like are expected.

The echo obtained by excitation using a half RF pulse is measurement data from one side from the origin when the slice axis of the k space is considered. For this reason, in the UTE imaging, a signal equivalent to a signal obtained when a full RF pulse is used is acquired by performing two measurements in which the polarity of the slice gradient magnetic field applied with a half RF pulse is changed and performing complex addition of signals (raw data) acquired by these two measurements.

### Citation List

[Patent Document 1] U.S. Pat. No. 5,025,216

[Patent Document 2] U.S. Pat. No. 5,150,053

### SUMMARY OF INVENTION

### Technical Problem

In the UTE imaging, the half RF pulse and the slice gradient magnetic field are set such that the application start time thereof are equal to each other and the application end time thereof are equal to each other. In practice, however, there is a possibility that the gradient magnetic field pulse will be applied in a state shifted from the ideal for the RF pulse due to an eddy current or the characteristic of a gradient magnetic field coil.

When the gradient magnetic field pulse is applied in a shifted state, the spin outside the original slice surface is excited. When the excitation pulse is a full RF pulse, this shift means the phase within the slice surface is not just refocused. In the UTE imaging, however, complex addition of a signal excited when the slice gradient magnetic field has a positive polarity and a signal excited when the slice gradient magnetic field has a negative polarity is performed. Accordingly, since a phase error caused by shift remains in the addition result, artifacts caused by an excitation signal outside the slice surface occur.

Moreover, in the UTE imaging, the slice gradient magnetic field is set to have positive and negative polarities for RF excitation as described above. Accordingly, at the off-center slice position, relative phase offset between them occurs. For this reason, if two signals measured by changing the polarity of the slice gradient magnetic field are complex-added as they are, artifacts occur.

It is an object of the invention to provide a method of measuring a phase error component equivalent to the shift of a slice gradient magnetic field from the ideal (setting value) and a method of correcting an application start time (GCdelay) of the slice gradient magnetic field on the basis of the measured phase error component. In addition, it is an object of the invention to provide a method of correcting a relative phase offset between two data items, which are measured by changing the polarity of the slice gradient magnetic field, as well.

### Solution to Problem

In the invention, in order to solve the problems described above, when executing the imaging pulse sequence using a high frequency magnetic field pulse with a partial waveform of a predetermined waveform, an application start time of a slice gradient magnetic field applied simultaneously with the high frequency magnetic field pulse is corrected. Specifically, an MRI apparatus of the invention has an imaging pulse sequence obtained by combination of first and second measurements. In the first measurement, a high frequency magnetic field pulse with a partial waveform of a predetermined waveform and a slice selection gradient magnetic field are applied. In the second measurement, a high frequency magnetic field pulse with a partial waveform of the predetermined waveform and a slice selection gradient magnetic field different from the slice selection gradient magnetic field of the first measurement are applied. The MRI apparatus is characterized in that a correction unit which corrects an application start time of the slice selection gradient magnetic field is provided. In addition, a pulse sequence adjusting method of the invention is an adjusting method of the imaging pulse sequence described above and is characterized in that it includes: a prescan step of acquiring a magnetic resonance signal for correcting the imaging pulse sequence by executing a prescan sequence; a correction step of correcting an application start time of a slice selection gradient magnetic field in the imaging pulse sequence using the magnetic resonance signal for correction; and a measurement step of executing the imaging pulse sequence by applying the slice selection gradient magnetic field with the corrected application start time.

In addition, a relative phase offset between two data items measured by changing the polarity of the slice gradient magnetic field is corrected.

In addition, shift (correction value of the application start time of the slice gradient magnetic field) of the slice gradient magnetic field is calculated from the magnetic resonance signal acquired by the prescan sequence (pre-measurement), and the application start time of the slice gradient magnetic field is corrected on the basis of the calculated correction value.

In addition, a relative phase offset between two magnetic resonance signals measured by the prescan sequence having slice gradient magnetic fields with different polarities is calculated, and the measurement data of the corresponding slice position measured by the imaging pulse sequence is corrected by removing the relative phase offset on the basis of the calculated correction value.

For example, the prescan sequence includes a first prescan sequence, in which a magnetic resonance signal is measured by applying a readout gradient magnetic field of the same axis as the slice gradient magnetic field after applying a high frequency magnetic field pulse and a slice gradient magnetic field, and a second prescan sequence, in which a magnetic resonance signal is measured by applying the same readout gradient magnetic field as in the first prescan sequence except that the slice gradient magnetic field applied simultaneously with application of the high frequency magnetic field pulse is different.

Alternatively, the prescan sequence includes a prescan sequence of measuring a magnetic resonance signal with a corresponding slice gradient magnetic field direction as a reading direction after application of high frequency magnetic field pulses of all waveforms and the slice gradient magnetic field. The prescan sequence is executed at least twice by changing the slice gradient magnetic field.

Alternatively, the prescan sequence is executed after a correction value is applied by making the high frequency magnetic field pulse equal to the high frequency magnetic field pulse used in the imaging pulse sequence. In this case, this prescan sequence is executed for the same slice number and the same slice position as in the imaging pulse sequence.

### ADVANTAGEOUS EFFECTS OF INVENTION

According to the MRI apparatus of the invention, since means for correcting the application start time (GCdelay) of the slice gradient magnetic field in the imaging pulse sequence and means for correcting the amount of relative phase offset between two signals excited by different slice gradient magnetic fields are provided, a good image with no artifact which is the same as in imaging using a full RF pulse can be obtained in UTE imaging using a half RF pulse.

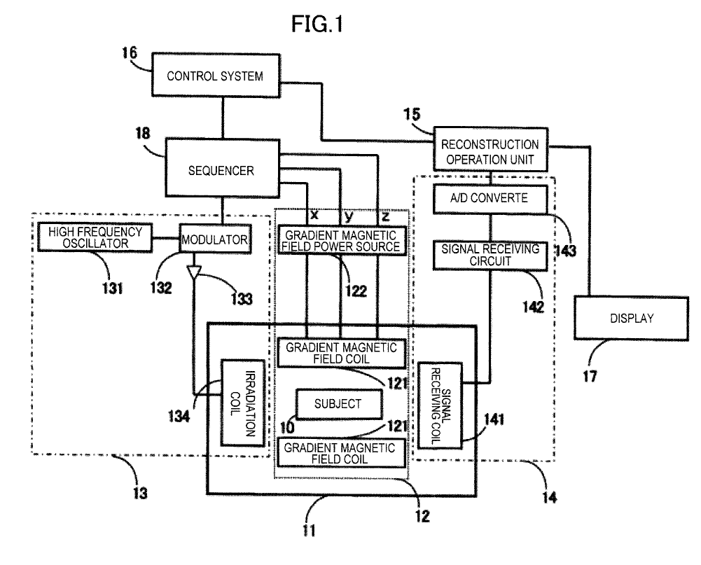

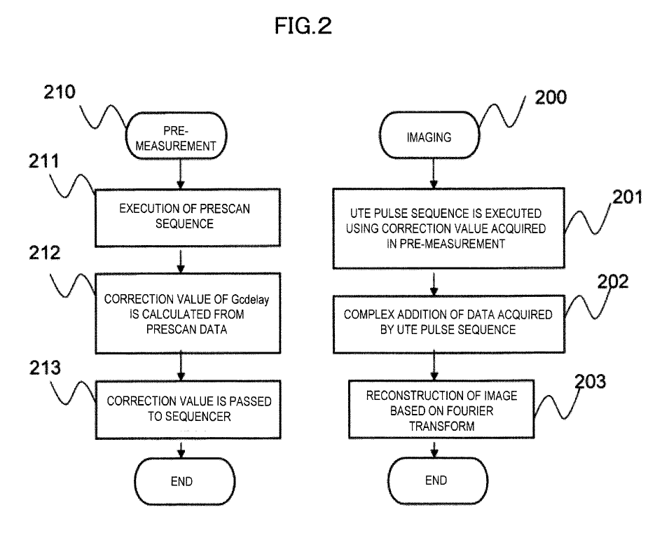

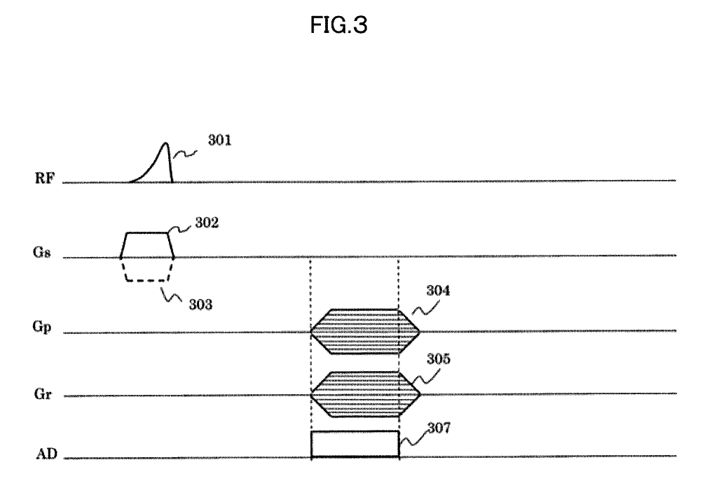

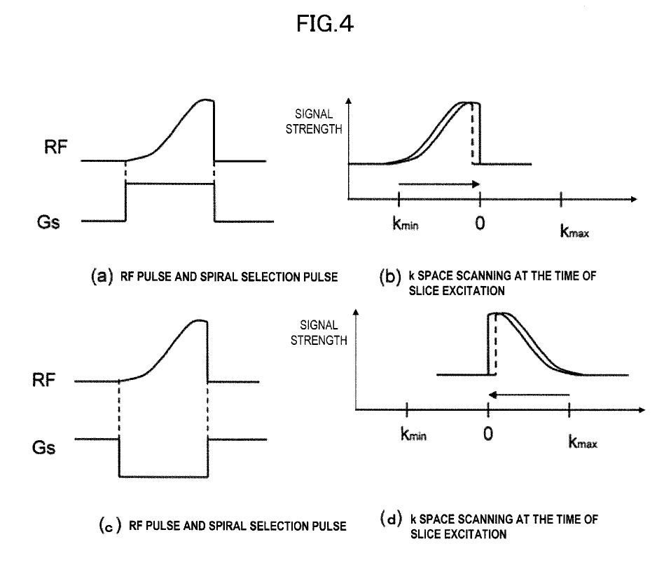

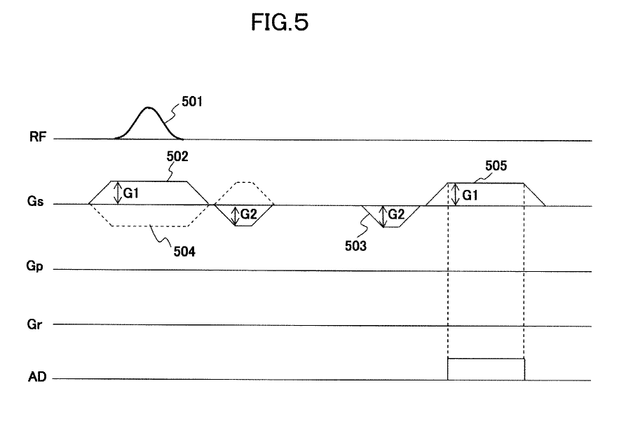

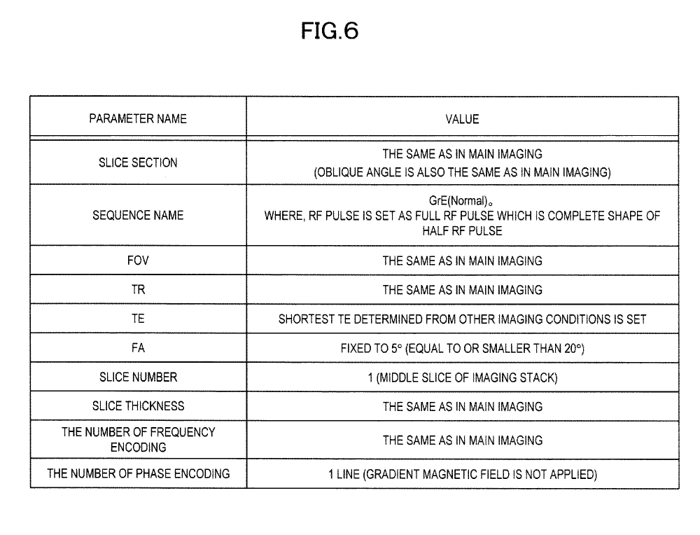

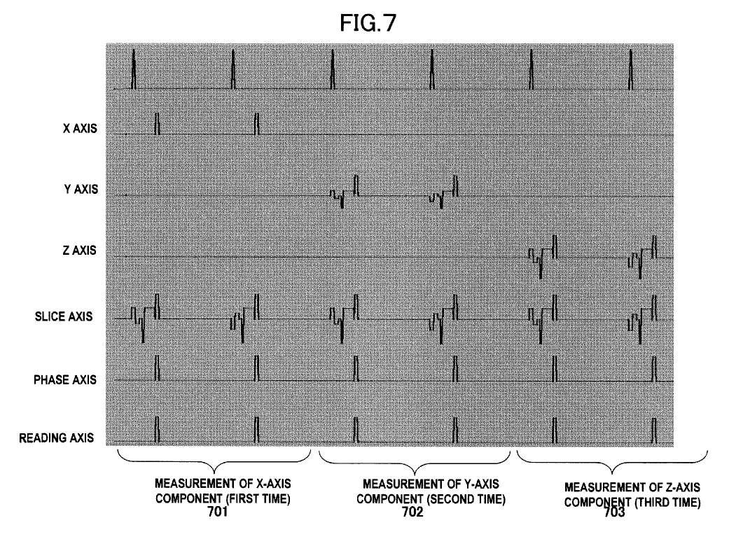

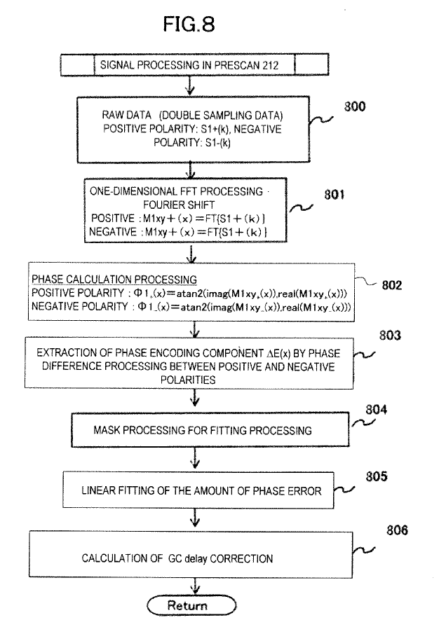

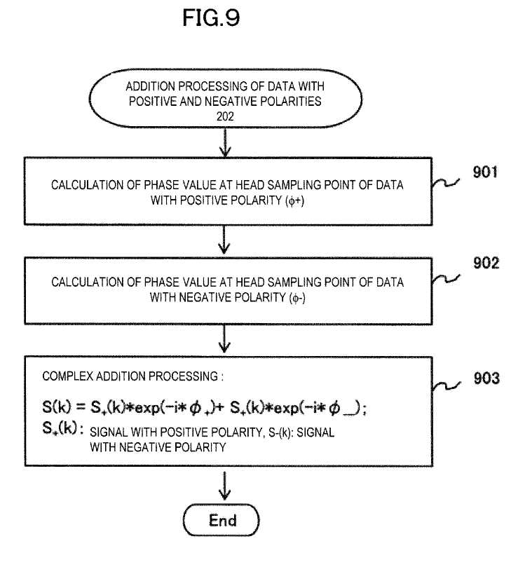

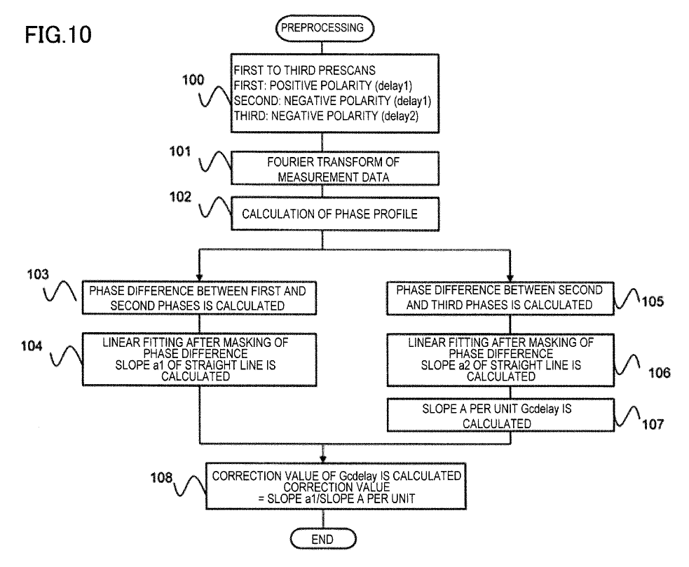

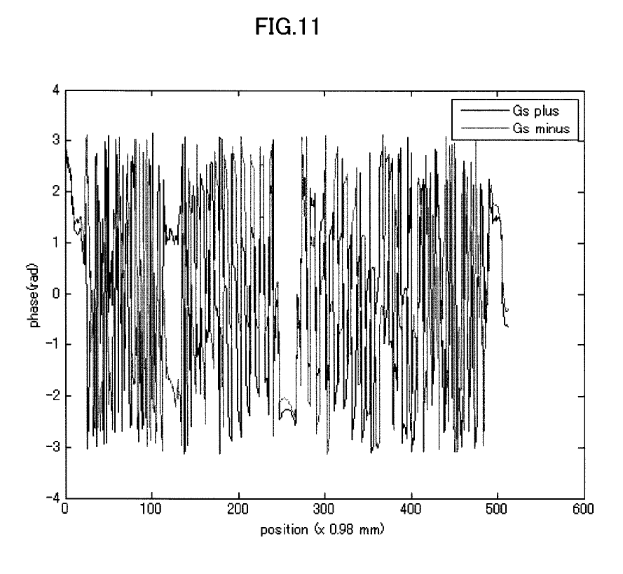

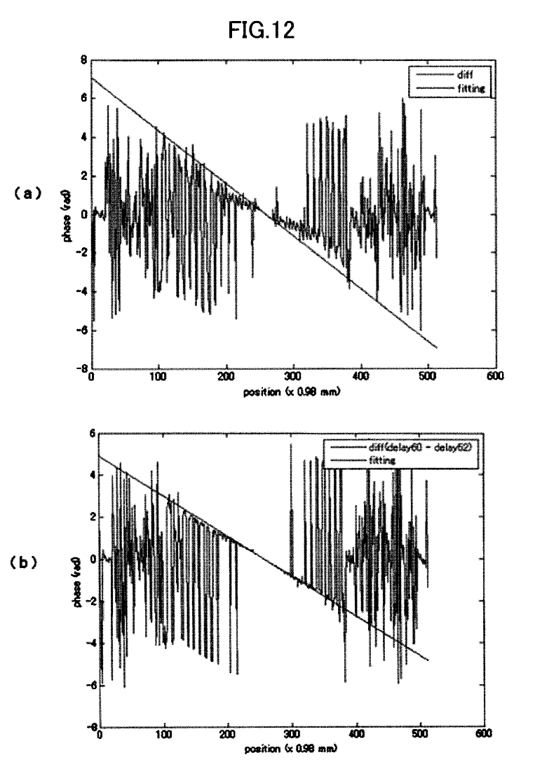

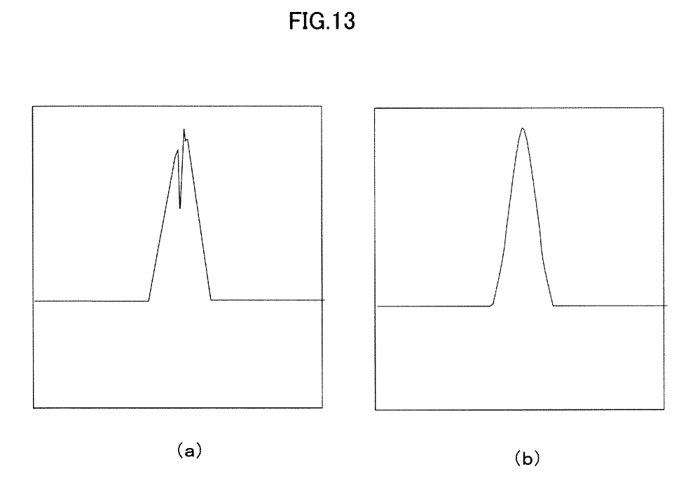

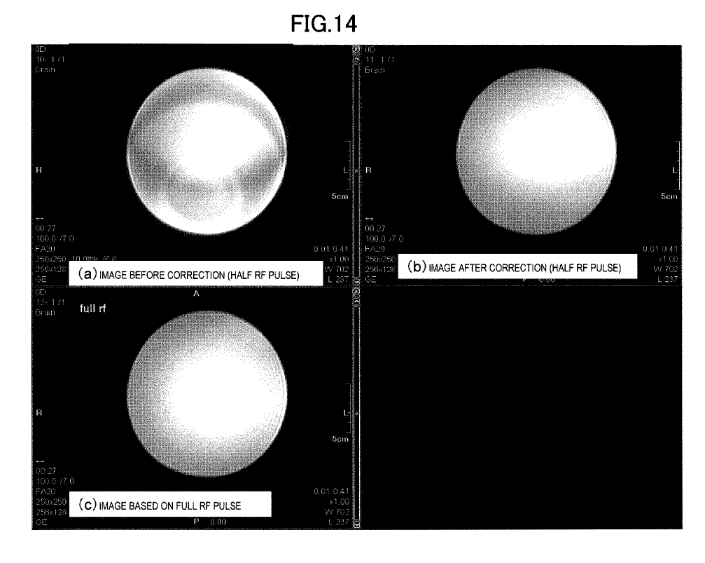

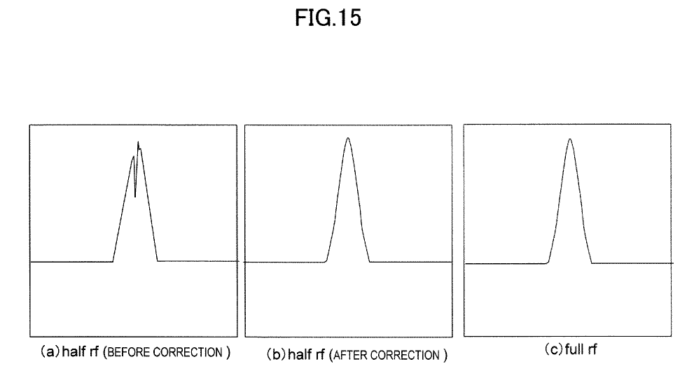

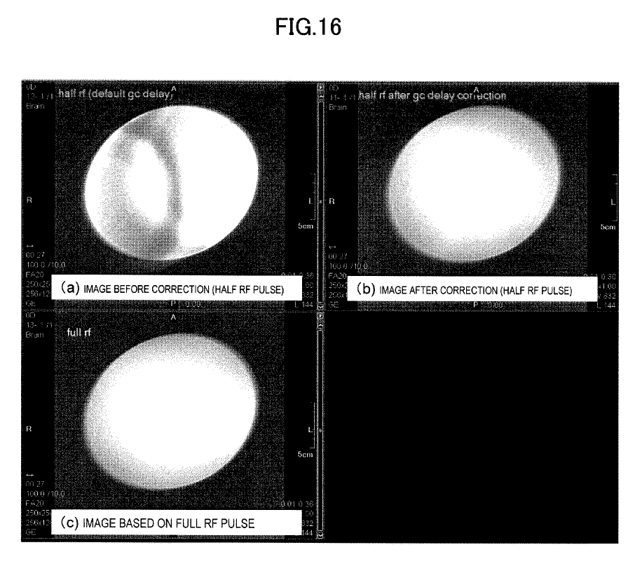

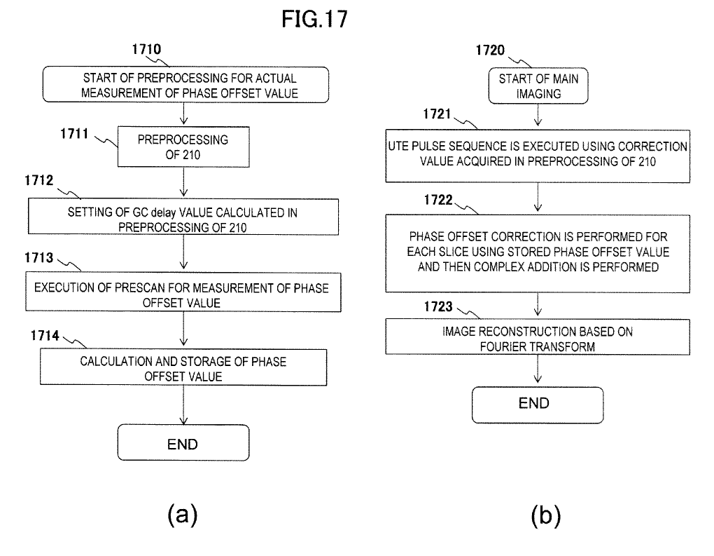

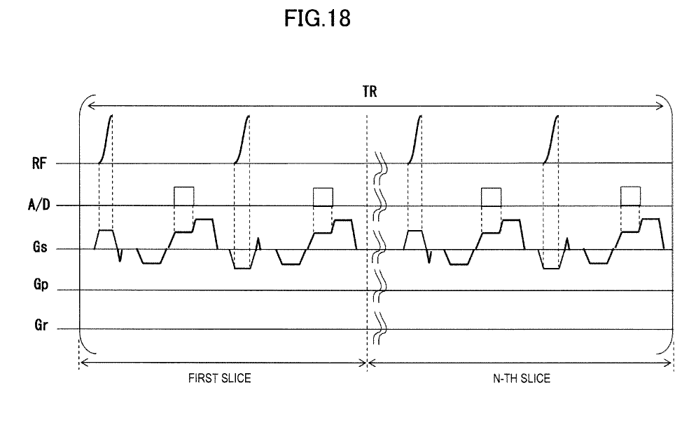

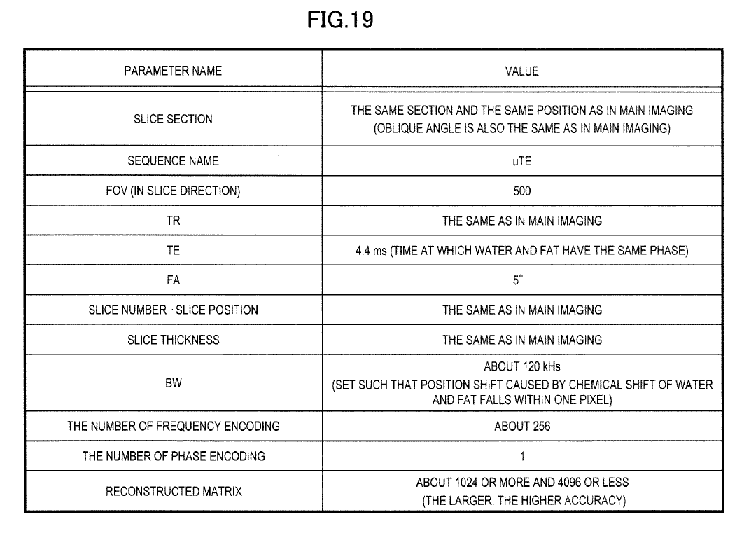

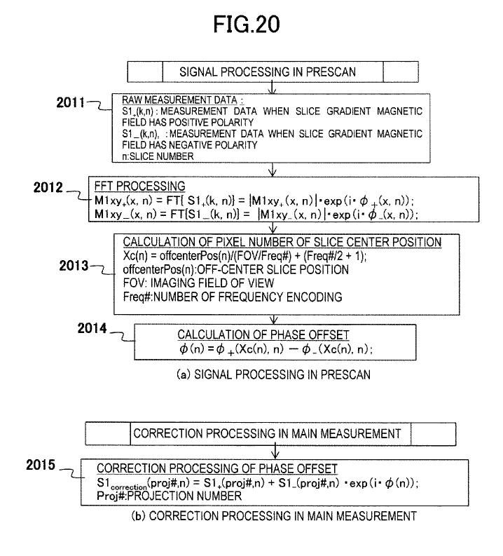

### BRIEF DESCRIPTION OF DRAWINGS

[FIG. 1] FIG. 1 is a view showing the outline of an entire MRI apparatus to which the invention is applied.

[FIG. 2] FIG. 2 is a view showing the imaging procedure using the MRI apparatus of the invention

[FIG. 3] FIG. 3 is a view showing an example of the UTE pulse sequence of the MRI apparatus of the invention.

[FIG. 4] FIG. 4 is a view showing k space scanning of a slice excited by the pulse sequence in FIG. 3.

[FIG. 5] FIG. 5 is a view showing an example of the pulse sequence of pre-measurement in a first embodiment.

[FIG. 6] FIG. 6 is a table showing parameters of the pulse sequence of preprocessing.

[FIG. 7] FIG. 7 is a view showing another example of the pulse sequence of pre-measurement in the first embodiment.

[FIG. 8] FIG. 8 is a view showing details of the procedure of preprocessing.

[FIG. 9] FIG. 9 is a view showing the procedure of signal processing performed in preprocessing.

[FIG. 10] FIG. 10 is a view showing the procedure of preprocessing in a second embodiment.

[FIG. 11] FIG. 11 is a view showing the phase profile of raw data obtained by preprocessing. Each is a phase profile of the raw data of first and second measurements.

[FIG. 12] FIG. 12 is a view showing a result obtained by taking the phase difference of the phase profiles in FIG. 11, where (a) is a phase difference of results of first and second measurements and (b) shows a phase difference of results of second and third measurements.

[FIG. 13] FIG. 13 is a view showing the k space signal profile obtained by imaging in a first example, where (a) shows “before correction” and (b) shows “after correction”.

[FIG. 14] FIG. 14 is a view showing images obtained by imaging in the first example, where (a) shows an image before correction (Half RF pulse), (b) shows an image after correction (Half RF pulse), and (c) shows an image based on a Full RF pulse.

[FIG. 15] FIG. 15 is a view showing the k space signal profile obtained by imaging in a second example, where (a) shows half RF (before correction), (b) shows half RF (after correction), and (c) shows full RF.

[FIG. 16] FIG. 16 is a view showing images obtained by imaging in the second example, where (a) shows an image before correction (Half RF pulse), (b) shows an image after correction (Half RF pulse), and (c) shows an image based on a Full RF pulse.

[FIG. 17] FIG. 17 is a view showing the procedure regarding phase offset correction in this MRI apparatus, where (a) shows the procedure in preprocessing and (b) shows the correction procedure in main measurement.

[FIG. 18] FIG. 18 is a view showing an example of the pulse sequence of preprocessing for measuring the phase offset value.

[FIG. 19] FIG. 19 is a table showing parameters of the pulse sequence of preprocessing for measuring the phase offset value.

[FIG. 20] FIG. 20 is a view showing the flow regarding calculation of the phase offset value and correction processing.

### DESCRIPTION OF EMBODIMENTS

Hereinafter, embodiments of the invention will be described.

FIG. 1 shows the entire configuration of an MRI apparatus to which the invention is applied. As shown in FIG. 1, the MRI apparatus mainly includes: a static magnetic field generating system 11 which generates a uniform static magnetic field around a subject 10; a gradient magnetic field generating system 12 which gives a magnetic gradient in three axial directions (x, y, and z) perpendicular to the static magnetic field; a high frequency magnetic field generating system 13 which applies a high frequency magnetic field to the subject 10; a signal receiving system 14 which detects a magnetic resonance signal generated from the subject 10; a reconstruction operation unit 15 which reconstructs a tomographic image, a spectrum, or the like of the subject using the magnetic resonance signal received by the signal receiving system 14; and a control system 16 which controls operations of the gradient magnetic field generating system 12, the high frequency magnetic field generating system 13, and the signal receiving system 14.

Although not shown, a magnet, such as a permanent magnet or a superconducting magnet, is disposed in the static magnetic field generating system 11, and the subject is placed in the bore of the magnet. The gradient magnetic field generating system 12 includes gradient magnetic field coils 121 in the three axial directions and a gradient magnetic field power source 122 which drives the gradient magnetic field coils 121. The high frequency magnetic field generating system 13 includes: a high frequency oscillator 131; a modulator 132 which modulates a high frequency signal generated by the high frequency oscillator 131; a high frequency amplifier 133 which amplifies a modulated high frequency signal; and an irradiation coil 134 which receives a high frequency signal from the high frequency amplifier 133 and irradiates the subject 10 with the high frequency magnetic field pulse.

The signal receiving system 14 includes: a signal receiving coil 141 which detects a magnetic resonance signal from the subject 10; a signal receiving circuit 142 which receives the signal detected by the signal receiving coil 141; and an A/D converter 143 which converts an analog signal received by the signal receiving circuit 142 into a digital signal at a predetermined sampling frequency. The reconstruction operation unit 15 performs operations, such as correction calculation and the Fourier transform, on the digital signal output from the A/D converter 143 in order to reconstruct an image. The processing result in the reconstruction operation unit 15 is displayed on a display 17.

The control system 16 controls the operation of the entire apparatus described above and in particular, includes a sequencer 18 for controlling the operations of the gradient magnetic field generating system 12, the high frequency magnetic field generating system 13, and the signal receiving system 14 at a predetermined timing determined by an imaging method and a storage unit (not shown) which stores a parameter required for control and the like. The timing of each magnetic field pulse generation controlled by the sequencer 18 is called a pulse sequence, and various kinds of pulse sequences are stored in the storage unit in advance. By reading and executing a desired pulse sequence, imaging is performed.

The control system 16 and the reconstruction operation unit 15 include user interfaces for a user to set the conditions or the like required for their internal processing. Through these user interfaces, selection of an imaging method or setting of a parameter required for execution of the pulse sequence is performed.

### First Example

A first embodiment of the invention will be described on the basis of the outline of the apparatus described above. The imaging procedure of the MRI apparatus according to the present embodiment is shown in FIG. 2. The MRI apparatus of the present embodiment is characterized in that pre-measurement (prescan) 210 for acquiring the correction data for correcting the conditions of the gradient magnetic field used in the main imaging is executed before imaging 200 for acquiring the image data of the subject. In the pre-measurement 210, a phase error is corrected after being measured from two signals, which are measured using the slice gradient magnetic fields with positive and negative polarities in full RF (high frequency magnetic field pulse with a predetermined waveform) excitation, using the fact that the relationship based on the Fourier transform is satisfied between the RF pulse function and the transverse magnetization Mxy excited thereby.

As characteristics of the Fourier transform, the “principle of Fourier shift” indicating that a position shift of the peak of a k space signal is equivalent to the slope of the phase of the real space is satisfied. Generally, the transverse magnetization Mxy occurring in excitation by an RF pulse follows the equation of Bloch. Here, if the RF pulse has a low flip angle FA (flip angle) which is equal to or smaller than about 20°, the relationship between the RF pulse and the transverse magnetization Mxy occurring by the RF pulse can be approximated satisfactorily by the relationship (linear transform) of the Fourier transform. In this case, the peak shift (shift of one peak position to the other peak position) of two k space signals measured in the slice gradient magnetic fields with the positive and negative polarities is equivalent to the slope of the phase in the real space, and follows the “principle of Fourier shift”. Therefore, in the pre-measurement 210, phase shift equivalent to the shift of a peak position is calculated from the data measured in the low FA conditions, shift of the peak position is converted from the calculated phase shift, and the correction value of the application start time GCdelay of the slice gradient magnetic field is eventually acquired by calculation.

Specifically, the pre-measurement 210 includes: a step 211 of executing a prescan sequence; a step 212 of calculating a phase shift from the measurement data acquired by the prescan and calculating the application time (correction value of GCdelay) of the gradient magnetic field from the phase shift; and a step 213 of passing the correction value to the sequencer which controls the imaging pulse sequence. The imaging 200 includes: a step 201 of executing a UTE pulse sequence (imaging pulse sequence) using the correction value acquired in the pre-measurement 210, that is, the correction value of the application time GCdelay of the slice gradient magnetic field; complex addition processing 202 on two sets of data acquired in the slice gradient magnetic fields with positive and negative polarities; and an image reconstruction step 203 using the data after complex addition.

FIG. 3 shows an example of the UTE pulse sequence. As shown in FIG. 3, in UTE imaging, a half-wave (partial waveform of a predetermined waveform) high frequency (RF) pulse 301 is applied together with a slice gradient magnetic field pulse 302, and then readout gradient magnetic field pulses 304 and 305 are applied and an echo signal is measured simultaneously with the application. In the drawing, an A/D 307 indicates a sampling time of an echo signal. The UTE pulse sequence is characterized in that a refocusing pulse of the slice gradient magnetic field pulse 302 is not used. Accordingly, the measurement 307 of a signal in the very short TE becomes possible. As shown in the drawing, the slice refocusing pulse is not generally used. However, it is a matter of course that the refocusing pulse may be used. In the example shown in the drawing, the readout gradient magnetic field pulse is measured from the rising edge without using a dephasing gradient magnetic field (non-linear measurement). In the invention, however, the dephasing gradient magnetic field may also be used. However, in order to shorten the TE time which is the characteristic of the UTE imaging, the dephasing gradient magnetic field is not usually used.

Then, the polarity of the slice gradient magnetic field pulse 302 is inverted (pulse 303 is applied), and other than that the same pulse sequence as the pulse sequence shown in FIG. 3 is repeated. The situation of k space scanning in the slice direction at the time of slice excitation in these two measurements is shown in FIG. 4. In this drawing, (a) and (b) of FIG. 4 show the case where the slice gradient magnetic field with a positive polarity is applied, and (c) and (d) of FIG. 4 show the case where the slice gradient magnetic field with a negative polarity is applied. (a) and (c) of FIG. 4 show the relationship between an RF pulse and a slice selection pulse, and (b) and (d) of FIG. 4 show the situation of k space scanning at the time of slice excitation.

As shown in the drawings, a range from the left end (—kmin) of the kz axis of the k space to the origin is scanned when the slice gradient magnetic field with a positive polarity is applied, and a range from the right end (—kmax) of the kz axis of the k space to the origin is scanned when the slice gradient magnetic field with a negative polarity is applied. Therefore, performing complex addition of these becomes the same as scanning the range from the left end of the kz axis to the right end. Since the final point after scanning is ideally the origin, the phase in the slice direction is refocused.

Here, when the slice gradient magnetic field 303 is shifted from the RF pulse, that is, when the calculated value (application start time, strength) of the slice gradient magnetic field and the slice gradient magnetic field actually applied are shifted from each other, scanning is performed so as to be shifted from the origin of the k space as shown by a dotted line in (d) of FIG. 4. This shift can be solved by correcting the application start time GCdelay of the gradient magnetic field. Therefore, in the pre-measurement 210, this correction value is measured.

Hereinafter, each processing of the pre-measurement 210 will be described in detail.

<<Step 211>>

Here, in order to calculate a phase shift, a prescan sequence is executed and an echo signal is measured. An example of the prescan sequence is shown in FIG. 5, and an example of the parameter is shown in FIG. 6. Generally, if an imaging pulse sequence is selected, the parameters TE, TR, FOV, and the like are set in a sequencer by designation of a user or as a default value. In the pre-measurement 210, a parameter of the prescan sequence is set with reference to the parameter of the imaging 200.

As shown in FIG. 5, the prescan sequence is a pulse sequence based on the normal 2D gradient echo system. In this prescan sequence, a slice gradient magnetic field pulse 502 is applied simultaneously with an RF pulse 501, readout gradient magnetic field pulses 503 and 505 with inverted polarities are then applied, and a gradient echo occurring during the application of the readout gradient magnetic field pulse 505 is measured.

The RF pulse 501 is a full RF pulse having a symmetric function as an envelope, and its application time is set to twice the application time of a half RF pulse used in the UTE pulse sequence which is an imaging sequence. Since the relationship of the Fourier transform is satisfied between an RF pulse and transverse magnetization excited by the RF pulse, it is preferable that the flip angle of the RF pulse is as small as possible so that it is within the range where the principle of Fourier shift can be satisfied. For example, the flip angle of the RF pulse is set to 20° or less, more preferably about 5°.

The slice gradient magnetic field applied simultaneously with an RF pulse is set to have the same axis, the same strength G1, and the same slew rate as the slice gradient magnetic field used in the imaging pulse sequence. This is because the shift is different if the axis and the strength are different. The strength G2 of the refocusing gradient magnetic field and the strength G2 of the dephasing gradient magnetic field are also the same. In addition, since the slice refocusing gradient magnetic field may not be used in the UTE imaging of the main imaging, it is preferable that the strength and the slew rate of the refocusing gradient magnetic field are low. In the case of oblique imaging, a combination of an axis and a strength at which the same oblique angle as in imaging is obtained is set. In addition, the slice thickness is set to the same thickness as in the imaging. The phase encoding gradient magnetic field is not used.

The readout gradient magnetic fields 503 and 505 are set to have the same axis as the slice gradient magnetic field 502, and the echo time TE is set as the shortest TE determined from the other imaging conditions. Preferably, the application timing is set to TE at which water and fat have the same phase. In the measurement of an echo, FOV is made to be equal to FOV of the imaging. In the present embodiment, the measurement data is used as double sampling data. Then, the polarity of the slice gradient magnetic field 502 is inverted, and the same pulse sequence is executed without changing the polarities of the readout gradient magnetic fields 503 and 505 in order to measure an echo. This repetition time TR is set to be equal to TR of the imaging pulse sequence.

Measurement having two measurements (measurement of a positive polarity and measurement of a negative polarity), which are performed while changing the polarity of the slice gradient magnetic field, as one set is performed. When the imaging section is an oblique surface, this is executed for each of the gradient magnetic field components in three orthogonal directions (X, Y, and Z) which are obliquely expanded, as shown in FIG. 7. The measurement data acquired in one to three sets of prescan 701 to 703 is used to calculate a phase shift in the next step 212.

<<Step 212>>

In step 212, among phase errors included in each of the data acquired by two measurements, a phase error component regarding the gradient magnetic field in the slice direction is acquired by calculation. Details of the processing performed in step 212 are shown in FIG. 8.

A signal measured by applying the slice gradient magnetic field with a positive polarity is set as S1₊(k), and a signal measured by applying the slice gradient magnetic field with a negative polarity is set as S1₋(k) (step 800). By one-dimensional Fourier transform of these signals, image space data M1xy₊ and M1xy₋ are acquired (step 801). The phases φ1₊(x) and φ1₋(x) of the image space data (complex data) are calculated by the following Expressions (1) and (2) (step 802).

φ1₊(x)=atan2(imag(M1xy₊(x)), real(M1xy₊(x))) (1)

φ1₋(x)=atan2(imag(M1xy₋(x)), real(M1xy₋(x))) (2)

In Expressions, x is a pixel number in the image space. As phase error components included in the phases φ1₊(x) and φ1₋(x), there are phase error components with different phase polarities (components shifted in different directions in the k space) and phase error components occurring with the same phase polarity (components shifted in the same direction in the k space). The former is a phase error component occurring in an eddy current or the like and is a phase error calculated in this processing, and the latter is a phase error occurring due to non-uniformity of the static magnetic field or offset shift of the gradient magnetic field. Assuming that phase error components with different polarities are ΔE(x) and all phase error components with the same polarity are ΔB(x), the phases φ1₊(x) and φ1₋(x) can be expressed as Expressions (3) and (4), respectively.

φ1₊(x)=ΔB(x)+ΔE(x) (3)

φ1₋(x)=ΔB(x)−ΔE(x) (4)

Since ΔB(x) is eliminated by differential processing of the phases φ1₊(x) and φ1₋(x) with positive and negative polarities, the phase error component ΔE (x) can be calculated (step 803). That is, the phase error component ΔE(x) can be calculated by Expression (5).

ΔE(x)=(φ1₋(x)−φ1₊(x))/2 (5)

Since this phase error is equivalent to the slope of the phase of image space data, the slope is calculated by linear fitting of the phase error component (step 805). Before the linear fitting, mask processing of the image space data is performed in order to improve the fitting accuracy (step 804). For example, the mask processing is performed by creating a mask image Mask(x), in which the absolute value of the image space data M1xy₊ equal to or larger than 50% of the maximum value is set to 1 and the absolute value of the image space data M1xy₊ smaller than 50% of the maximum value is set to 0, and, as expressed in Expression (6), multiplying ΔE (x) by this mask image.

ΔE′(x)=ΔE(x)×Mask(x) (6)

By performing linear fitting processing of ΔE′(x) after masking, Expression (7) is obtained.

ΔE′(x)=a×(±π/(2×FOV)×x+b×2π (7)

In this Expression, FOV is a field-of-view size. The first-order coefficient a of Expression (7) is a phase error component to be calculated and is equivalent to the shift amount of the peak position of the k space. The shift amount of the peak position of the k space can be converted into the amount of time lag, that is, the amount of correction At of GCdelay by the following Expression (8) (step 806).

Δt (ΔGCdelay)=a×(sampling time of a k space signal)=a×1/(2×BW) (8)

In this Expression, BW is a received signal bandwidth. The reason why the denominator is set to 2×BW is that signals of the k space are double sampling data.

The correction value calculated in this way in step 212 is passed to the sequencer, and the GCdelay (default value) of the slice axis in the imaging pulse sequence is replaced with the GCdelay value after correction. In addition, when performing prescan in the three axial directions as shown in FIG. 7, the above-described step 212 is performed for three sets of pre-measurement data and the correction value of each axis is passed to the sequencer.

In the imaging 200, the UTE pulse sequence is executed using the correction value of GCdelay calculated in step 212 in order to measure the data (echo) for an image (step 201). When the UTE pulse sequence includes phase encoding, a set of (positive and negative) data is obtained every phase encoding by repeating data measurement using the slice gradient magnetic field with a positive polarity and data measurement using the slice gradient magnetic field with a negative polarity while changing the phase encoding.

When the UTE pulse sequence is a non-linear measurement in which the phase encoding shown in FIG. 3 is not used, measurement data which spreads radially from the origin of the k space is obtained by repeating measurement while changing the strength of the readout gradient magnetic field. A set of measurement data is obtained by performing such measurement for both positive and negative polarities of the slice gradient magnetic field.

Then, the measurement data is processed and complex addition of a set of measurement data is performed to create the k space data (step 202). In the case of measurement using phase encoding, one data item along the horizontal axis of the k space is created by complex addition of the data measured by applying the slice gradient magnetic field with a positive polarity and the data measured by applying the slice gradient magnetic field with a negative polarity. Data which fills the k space is obtained by performing complex addition for all measurement data based on different phase encoding. In the case of data obtained by non-linear measurement, complex addition of the radial data is performed at the same angle and then coordinate transformation (gridding) is performed to set the k space data.

Specifically, in the addition processing, the phase values φ₊ and φ₋ at the head sampling point of data are calculated first for each of the data S₊(k) when the slice gradient magnetic field has a positive polarity and the data S₋(k) when the slice gradient magnetic field has a negative polarity, as shown in FIG. 9 (steps 901 and 902). Then, the complex addition is performed using Expression (9) (step 903).

S(k)=S₊(k)×exp(−i×φ₊)+S₋(k)×exp(−i×φ₋) (9)

The image data is obtained by Fourier transform of the k space data after complex addition (step 203).

Although the correction of the phase offset values φ₊ and φ₋ in Expression (9) was described using the simple method in the above, it is preferable to execute pre-measurement for measuring the phase offset value and to correct it using the correction value (phase offset value) actually measured.

Hereinafter, preprocessing 1710 for actual measurement of the phase offset value will be described in detail using (a) of FIG. 17.

<<Steps 1710 to 1712>>

Here, in order to calculate a phase offset, a prescan sequence in which the GCdelay correction value calculated in the preprocessing (step 1711) of 210 described above is applied is executed (steps 1712 and 1713), and an echo signal is measured. An example of the prescan sequence is shown in FIG. 18, and an example of the parameter at that time is shown in FIG. 19. Generally, if an imaging pulse sequence is selected, the parameters TE, TR, FOV, and the like are set in a sequencer by designation of a user or as a default value. In the preprocessing 1710, a parameter of the prescan sequence is set with reference to the imaging parameter.

As shown in FIG. 18, the prescan sequence is a pulse sequence based on the normal 2D gradient echo system. In this prescan sequence, a slice gradient magnetic field pulse is applied simultaneously with an RF pulse and then a dephasing pulse of the readout gradient magnetic field is applied and a readout gradient magnetic field pulse is applied continuously, and a gradient echo occurring during the application is measured.

As the RF pulse, the same half RF pulse as in the main imaging is used. Since the relationship of the Fourier transform is satisfied between an RF pulse and transverse magnetization excited by the RF pulse, it is preferable that the flip angle of the RF pulse is as small as possible so that it is within the range where the principle of Fourier shift can be satisfied. For example, the flip angle of the RF pulse is set to 20° or less, more preferably about 5°. As the excitation frequency, the same frequency as in the main imaging is used so that the same imaging surface and the same slice position as in the main imaging are excited.

The slice gradient magnetic field applied simultaneously with an RF pulse is set to have the same axis, the same strength, and the same slew rate as the slice gradient magnetic field used in the imaging pulse sequence. This is because the phase offset values to be measured are different if the axis and the strength are different. The strength of the slice refocusing gradient magnetic field is also the same. In the case of oblique imaging, the same oblique angle as in the main imaging is set. In addition, the slice thickness is set to the same thickness as in the imaging. The phase encoding gradient magnetic field is not used.

The readout gradient magnetic field is set to have the same axis as the slice gradient magnetic field, and the echo time TE is set as the shortest TE determined from the other imaging conditions. Preferably, the application timing is set to TE at which water and fat have the same phase.

Then, the polarity of the slice gradient magnetic field is inverted, and the same pulse sequence is executed without changing the polarity of the readout gradient magnetic field in order to measure an echo. This repetition time TR is set to be equal to TR of the imaging pulse sequence.

Measurement having two measurements (measurement of a positive polarity and measurement of a negative polarity), which are performed while changing the polarity of the slice gradient magnetic field, as one set is performed once per slice position, and this measurement is performed for all slice positions.

<<Step 1714>>

In step 1714, from the data acquired by two measurements per slice position, a difference of both phase offsets at the slice center position is calculated. Details of the processing performed in step 1714 are shown in FIG. 20.

A signal measured by applying the slice gradient magnetic field with a positive polarity is set as S1₊(k), and a signal measured by applying the slice gradient magnetic field with a negative polarity is set as S1₋(k) (step 2011). By one-dimensional Fourier transform of these signals, image space data M1xy₊(x, n) and M1xy₋(x, n) are acquired (step 2012). The phases φ₊(x, n) and φ₋(x, n) of the image space data (complex data) are calculated by (1) and (2) of [Expression 1].

Then, the pixel number xc(n) of the slice center position in each slice is calculated by the following Expression (16) using the slice position offcenterPos(n), an imaging field of view FOV, and the number of frequency encoding Freq # of an arbitrary slice number n.

Xc(n)=OffcenterPos(n)/(FOV/Freq#)+(Freq#/2+1) (16)

In this Expression, OffcenterPos(n) is a slice position in the n-th slice, FOV is an imaging field of view, and Freq# is the number of frequency encoding.

Finally, for one slice position n, the phase difference at the position of Xc(n) is calculated from Expression (17) using two items of the data M1xy₊(x, n) and M1xy₋(x, n) measured in the slice gradient magnetic fields with positive and negative polarities. The value calculated herein is a phase offset value at this slice position.

φ(n)=φ₊(Xc(n), n)−φ₋(Xc(n), n) (17)

This calculation is performed for all slices, and the results are stored.

<<Step 1721>>

This is the same as step 201.

<<Step 1722>>

Step 1722 is a step of correction processing in this measurement. In step 1722, a phase offset is corrected using Expression (18) for the data imaged in this measurement using the phase offset value φ(n) stored in preprocessing. After correcting all data of one slice by performing correction for each projection, image reconstruction processing is performed.

(proj#, n) after S1 correction=S1₊(proj#, n)+S1₋(proj#, n)·exp(i*φ(n)) (18)

In this Expression, proj# is a projection number in UTE measurement, and n is a slice number.

In addition, since Half RF excitation itself is low in slice selectivity, magnetization of another slice position is excited even when a region deviated from the subject is excited as the slice center. As a result, a signal is generated. For this reason, it is preferable to determine from the signal strength whether or not the slice center position is a region deviated from the subject. When a region deviated from the subject is excited, it is preferable to set a blank image (zero value image) without performing correction based on Expression (18).

For example, assuming that the maximum signal value in the x direction at each slice position is PeakValue (n) and the maximum value of the maximum signal values at all slice positions is MaxSignal, it is determined that there is no subject at the position if Expression (19) is satisfied.

PeakValue(n)/MaxSignal<0.05 (19)

Although the threshold value was set to 0.05 herein, the threshold value may be strictly set to 0.1.

According to the present embodiment, by performing UTE imaging using GCdelay of the slice gradient magnetic field corrected on the basis of preprocessing, the shift between a half RF pulse and each of the slice gradient magnetic fields with positive and negative polarities can be removed and the phase offset value can also be corrected. As a result, it is possible to obtain the same good image as an image obtained when a full RF pulse is used.

According to the present embodiment, since the optimal correction value can be measured according to various imaging conditions set by the user, stable RF excitation becomes possible regardless of the conditions.

### Second Example

Also in the present embodiment, performing pre-measurement before imaging and calculating the phase shift between the case when the slice gradient magnetic field with a positive polarity is used and the case when the slice gradient magnetic field with a negative polarity is used from the data acquired in the pre-measurement using the principle of Fourier shift and calculating the application start time GCdelay of the gradient magnetic field are the same as in the first embodiment. However, although GCdelay equivalent to a phase error part was calculated by Expression (8) using the signal receiving bandwidth BW in the first embodiment, the phase shift per unit GCdelay is calculated by performing two or more measurements with different GCdelay as pre-measurements in the present embodiment.

The procedure of the second embodiment is shown in FIG. 10. First, a prescan pulse sequence is executed. The prescan pulse sequence is the same as that shown in FIG. 5. The parameters (slice thickness, TR, FOV, and the like) are the same as those in the imaging pulse sequence, and a full RF pulse is used an RF pulse. In the present embodiment, however, a prescan (third prescan) with an application start time GCdelay of the slice gradient magnetic field, which is different from the first and second prescans, is performed in addition to the prescan (first prescan) using a pulse with a positive polarity as the slice gradient magnetic field and the prescan (second prescan) using a pulse with a negative polarity (step 100). In the third prescan, the polarity of the slice gradient magnetic field may be either a positive polarity or a negative polarity. In the present embodiment, the case of using a pulse with a negative polarity will be described.

Signals acquired in the first to third prescans are set as real space data by the Fourier transform, and phase profiles are calculated by Expressions (1) and (2) used in the first embodiment (steps 101 and 102). Then, from these phase profiles, a phase error component is acquired by the following calculation (steps 103 to 107).

Assuming that the phase profiles of signals (real space data) acquired in the first to third prescans are φ1₊(x), φ1₋(x), and φ2₋(x), respectively, they are expressed by the following Expressions.

φ1₊(x)=ΔB(x)+ΔE(x) (3)

φ1₋(x)=ΔB(x)−ΔE(x) (4)

φ2₋(x)=ΔB(x)−ΔE(x)+ΔD(x) (10)

Expressions (3) and (4) are the same as Expressions (3) and (4) of the first embodiment, ΔB(x) and ΔE(x) indicate the same phase error. By taking the phase difference of φ1₊(x) and φ1₋(x) (Expression (5)), the phase error component ΔE(x) with a different polarity is calculated (step 103). After mask processing of ΔE(x), linear fitting is performed (Expression (11)) to calculate the slope a1 (step 104).

ΔE(x)=(φ1₋(x)−φ1₊(x))/2 (5)

ΔE(x)=a1(±π/(2×FOV))x+b1×2π (11)

On the other hand, ΔD(x) at the right side of Expression (10) is a phase error component occurring by changing GCdelay and can be calculated by taking the difference between φ1₋(x) and φ2₋ (x) using Expression (12) (step 105). Also for the phase error component ΔD(x), linear fitting is performed after mask processing in order to calculate the slope a2 of the obtained straight line (Expression (13)), similarly to ΔE(x) (step 106). By dividing this slope a2 by the difference between GCdelay (referred to as delay1) of the first and second measurements and GCdelay (referred to as delay2) of the third measurement (Expression (14)), a phase error component A per unit GCdelay is calculated (step 107).

ΔD(x)=φ2₋(x)−φ1₋(x) (12)

ΔD(x)=a2(±π/(2×FOV))x+b2×2π (13)

A=a2(delay1−delay2) (14)

In addition, by dividing the slope al calculated by Expression (11) by the slope A per unit calculated by Expression (14) (Expression (15)), the amount of correction Δdelay of GCdelay equivalent to al can be calculated (step 108).

Δdelay=a1/A (15)

The amount of correction Adelay of GCdelay calculated in this way is passed to the sequencer, and the imaging pulse sequence is executed with the corrected GCdelay (default GCdelay+Δdelay). This is the same as in the first embodiment, and the procedure of imaging is the same as that in the first embodiment. When the imaging is for an oblique surface, the above-described prescan is performed for three axes of X, Y, and Z to calculate each amount of correction of GCdelay.

Although the present embodiment has a different method of calculating the amount of correction Δdelay of GCdelay, the same effects as in the first embodiment can be acquired.

In addition, according to the present embodiment, a measurement error caused by prescan for correction can also be absorbed since a response of an actual phase when changing GCdelay by prescan for main correction can be seen.

### Other Embodiments

In the first and second embodiments, the case of performing imaging by calculating a shift of the slice gradient magnetic field by performing pre-measurement for the subject, which is an object to be imaged, and correcting the slice gradient magnetic field GCdelay on the basis of the shift at the time of main imaging has been described. However, the shift of the slice gradient magnetic field may be calculated in advance by apparatus characteristic measurement using a phantom instead of being calculated by pre-measurement for the subject.

In this case, measurement using a full RF pulse shown in FIG. 5 is performed at least twice for one axis by changing the strength of the slice gradient magnetic field (GC) using a phantom, and the amount of phase error per unit GC strength is calculated from the peak position shift between the profiles of the obtained measurement data. This measurement is performed at least two positions in one-axis direction, basically, at symmetric positions with respect to the origin, and the amount of phase error per unit GC strength is calculated similarly. Using the amount of phase error per unit GC strength at the two positions, the [amount of phase error per unit GC strength] per unit position is calculated. The gradient magnetic field characteristics can be acquired by performing this processing in three orthogonal axial directions.

The acquired gradient magnetic field characteristics are stored in a memory and are referred to at the time of imaging. They are converted into appropriate correction values according to the imaging conditions and are used for correction of GCdelay of the slice gradient magnetic field. Specifically, it can be corrected by calculating the amount of phase error at the position from the imaging slice position and the slice gradient magnetic field strength determined by imaging conditions and setting it in a sequence.

### Example of Imaging in the First Example

Using a cylindrical phantom, pre-measurement and imaging based on the first embodiment were performed. The imaging was performed using a UTE pulse sequence (half RF pulse) and imaging parameters of FOV=250 mm, TR/TE/FA=100 ms/7 ms/20°, slice thickness=10 mm, the number of frequency encoding/the number of phase encoding=256/128, and BW=48 kHz (BW where the same readout gradient magnetic field strength as the slice gradient magnetic field strength of the slice thickness of 10 mm is obtained). In the pre-measurement, a first measurement using a slice gradient magnetic field with a positive polarity, a second measurement using a slice gradient magnetic field with a negative polarity, and third measurement using a slice gradient magnetic field with a negative polarity and a different GCdelay from the first and second measurements were performed using a 2D GE pulse sequence (full RF pulse) shown in FIG. 5. The GCdelay of each of the first and second measurements was 52 [us] which was a default value, and the GCdelay of the third measurement was 60 [us]. The parameters were the same parameters as imaging parameters (however, phase encoding is not used), and the same imaging section (section perpendicular to the z axis) was used.

The result is shown in FIGS. 11 to 14. FIG. 11 shows a phase profile (equivalent to φ1₊(x) and φ1₋(x) of Expressions (3), (4), and (10)) of data (image space data) obtained by first and second prescans (positive polarity (delay1) and negative polarity (delay1)).

(a) of FIG. 12 shows a result (equivalent to ΔE(x) in Expression (5)) after taking the phase difference between the data of first prescan and the data of second prescan (phase difference between the positive and negative polarities), and (b) of FIG. 12 shows a result (equivalent to ΔD(x) in Expression (12)) after taking the phase difference between the data of second prescan and the data of third prescan (phase difference between different GCdelay).

The slope (a1) of the straight line after straight line fitting of the phase difference ΔE(x) shown in (a) of FIG. 12 was −2.2309 [×2π/FOV]. In addition, the slope (a2) of the straight line after straight line fitting of the phase difference ΔD(x) shown in (b) of FIG. 12 was −1.5530 [×2π/FOV], and the slope (A) per unit delay was −0.1941 [×2π/FOV] (=−1.5530÷8 (difference between two slice gradient magnetic fields GCdelay with a negative polarity)). From these values, the amount of shift Δdelay at the start of gradient magnetic field application which caused the slope of the phase equivalent to the phase amount of peak shift was calculated. The result was 11.49 [us] (=2.2309/0.1941).

As imaging, imaging (before correction) in which GCdelay was set to the default value 52 [us] and imaging (after correction) in which GCdelay was set to the value 64[us] (about 52+11.5) corrected using the correction value obtained by pre-measurement were performed. FIG. 13 is a schematic view of a k space signal profile of the measurement data obtained by two-time imaging. (a) is a view in which imaging was performed with GCdelay before correction, and (b) is a view in which a value after correction is used. Both (a) and (b) show the result of complex addition of the data using the slice gradient magnetic field with a positive polarity and the data using the slice gradient magnetic field with a negative polarity. FIG. 14 is a view showing an image created from the data after complex addition. (a) is a view in which imaging was performed with GCdelay before correction, and (b) is a view in which a value after correction is used. In addition, as a reference image, an image of measurement data imaged under the same conditions as the UTE pulse sequence except that a full RF pulse is used is shown in (c).

As can be seen from FIG. 13, distortion is found in a central portion of the signal profile in (a), but this distortion was removed by correcting the GCdelay in (b). In addition, as can be seen from FIG. 14, a signal from the outside of an original slice appears as an artifact before correction, but the artifact from the outside of the slice disappears after correction. As a result, the same good image as a reference image shown in (c) was obtained.

### Example of Imaging in the Second Example

Using a cylindrical phantom, pre-measurement and imaging (oblique imaging) based on the first embodiment were performed. The imaging was performed using a UTE pulse sequence (half RF pulse) and imaging parameters of FOV=250 mm, TR/TE/FA=100 ms/10 ms/20°, slice thickness=10 mm, the number of frequency encoding/the number of phase encoding=256/128, and BW=50 kHz. In the pre-measurement, in order to calculate a correction value of each GC axis of an oblique image, prescan using the slice gradient magnetic field with a positive polarity and prescan using the slice gradient magnetic field with a negative polarity were performed for each axis of the X, Y, and Z axes using the 2D GE pulse sequence (full RF pulse) shown in FIG. 7. In both the measurements, default values (X axis: 67 [us], Y axis: 72[us], and Z axis: 52[us]) were used as GCdelay. The parameters were the same parameters as in imaging (however, phase encoding is not used).

The phase profile of the real space data obtained by the Fourier transform of the measurement data obtained for the X, Y, and Z axes was calculated, and each phase difference between the positive and negative polarities was calculated. From the slope, GCdelay of the gradient magnetic field was calculated by Expression (8). As a result, the amount of correction of GCdelay was 14[us] (GCdelay after correction=81 [us]) for the X axis, 12 [us] (GCdelay after correction=84 [us]) for the Y axis, and 13[us] (GCdelay after correction=64 [us]) for the Z axis.

As the imaging, imaging in which GCdelay of each of the X, Y, and Z axes was set to the value before correction (default value) and imaging in which GCdelay of each of the X, Y, and Z axes was set to the value after correction were performed for the oblique section. The result is shown in FIGS. 15 and 16. FIG. 15 is a schematic view of the k space signal profile of measurement data, and shows a result of complex addition of data using a slice gradient magnetic field with a positive polarity and data using a slice gradient magnetic field with a negative polarity. FIG. 16 shows an image reconstructed from the data after complex addition. In both the drawings, (a) shows an imaging result before correction, (b) shows an imaging result after correction, and (c) is a result (reference) of imaging using a full RF pulse.

Also in this example, similarly to the first example, distortion ((a) of FIG. 15) of the central portion of the signal profile and artifacts ((a) of FIG. 16) from the outside of the slice, which were found before correction, disappeared through correction, and it was confirmed that the same result as the reference using a full RF pulse was obtained.

### REFERENCE SIGNS LIST

11: static magnetic field generating system
12: gradient magnetic field generating system
13: high frequency magnetic field generating system
14: signal receiving system
15: reconstruction operation unit
16: control system
17: display
18: sequencer

1. A magnetic resonance imaging apparatus comprising:
a gradient magnetic field generator;
a high frequency magnetic field pulse generator which generates a high frequency magnetic field pulse with a predetermined waveform;
a signal receiver which receives a magnetic resonance signal from a subject; and
a controller which controls each section on the basis of an imaging pulse sequence,
wherein the imaging pulse sequence is a combination of a first measurement and a second measurement,
in the first measurement, a high frequency magnetic field pulse with a partial waveform of the predetermined waveform and a slice selection gradient magnetic field are applied,
in the second measurement, a high frequency magnetic field pulse with a partial waveform of the predetermined waveform and a slice selection gradient magnetic field different from the slice selection gradient magnetic field of the first measurement are applied, and
a correction unit which corrects an application start time of the slice selection gradient magnetic field is provided.

2. The magnetic resonance imaging apparatus according to claim 1,
wherein the controller has a prescan sequence for measuring a magnetic resonance signal using the high frequency magnetic field pulse with the predetermined waveform, and
the correction unit calculates a correction value of the application start time of the slice selection gradient magnetic field in the imaging pulse sequence using the magnetic resonance signal acquired by the prescan sequence.

3. The magnetic resonance imaging apparatus according to claim 2,
wherein the prescan sequence includes a first prescan sequence, in which a magnetic resonance signal is measured by applying a readout gradient magnetic field of the same axis as the slice selection gradient magnetic field after applying the slice selection gradient magnetic field, and a second prescan sequence, in which the slice selection gradient magnetic field is different from that of the first prescan sequence, and
the correction unit calculates a correction value of the application start time of the slice selection gradient magnetic field in the imaging pulse sequence using magnetic resonance signals acquired by the first and second prescan sequences.

4. The magnetic resonance imaging apparatus according to claim 2,
wherein the correction unit calculates a correction value of the application start time of the slice selection gradient magnetic field in the imaging pulse sequence on the basis of a plurality of magnetic resonance signals acquired using a plurality of prescan sequences with different application start time of the slice selection gradient magnetic field.

5. The magnetic resonance imaging apparatus according to claim 2,
wherein the waveform of the high frequency magnetic field pulse applied in the prescan sequence is the same as the predetermined waveform.

6. The magnetic resonance imaging apparatus according to claim 2,
wherein the waveform of the high frequency magnetic field pulse applied in the imaging pulse sequence is approximately a half of the waveform of the high frequency magnetic field pulse applied in the prescan sequence.

7. The magnetic resonance imaging apparatus according to claim 2,
wherein a flip angle of the high frequency magnetic field pulse used in the prescan sequence is equal to or smaller than 20°.

8. The magnetic resonance imaging apparatus according to claim 2,
wherein an echo time (TE) used in the prescan sequence is a time at which nuclides of water and fat have the same phase.

9. The magnetic resonance imaging apparatus according to claim 2,
wherein the controller executes the prescan sequence at the same slice position as the imaging pulse sequence.

10. The magnetic resonance imaging apparatus according to claim 2,
wherein the controller sets a slice position excited by the prescan sequence as the approximate center of an excitation region in the imaging pulse sequence.

11. The magnetic resonance imaging apparatus according to claim 2,
wherein the controller executes the prescan sequence for each of gradient magnetic field directions of three axes perpendicular to each other.

12. The magnetic resonance imaging apparatus according to claim 3,
wherein the controller executes measurement based on the first prescan sequence and measurement based on the second prescan sequence for each of gradient magnetic field directions of three axes perpendicular to each other.

13. The magnetic resonance imaging apparatus according to claim 1, further comprising:
a storage unit which stores a parameter required for control of the controller,
wherein a correction value used by the correction unit is calculated from a plurality of magnetic resonance signals measured with a plurality of gradient magnetic field delay values using a phantom and is stored in the storage unit in advance, and
the correction unit uses the correction value stored in the storage unit.

14. The magnetic resonance imaging apparatus according to claim 3,
wherein the correction unit measures an amount of relative phase offset between magnetic resonance signals, which is caused by different slice selection gradient magnetic fields in the imaging pulse sequence, using magnetic resonance signals acquired using the first and second prescan sequences in which the application start time of the slice selection gradient magnetic field is corrected on the basis of the correction value.

15. An adjusting method of an imaging pulse sequence obtained by combination of a first measurement in which a high frequency magnetic field pulse with a partial waveform of a predetermined waveform and a slice selection gradient magnetic field are applied and a second measurement in which a high frequency magnetic field pulse with a partial waveform of the predetermined waveform and a slice selection gradient magnetic field different from the slice selection gradient magnetic field of the first measurement are applied, the pulse sequence adjusting method comprising:
a prescan step of acquiring a magnetic resonance signal for correcting the imaging pulse sequence by executing a prescan sequence;
a correction step of correcting an application start time of a slice selection gradient magnetic field in the imaging pulse sequence using the magnetic resonance signal for correction; and
a measurement step of executing the imaging pulse sequence by applying the slice selection gradient magnetic field with the corrected application start time.

16. The pulse sequence adjusting method according to claim 15,
wherein in the prescan sequence, a magnetic resonance signal is measured using the high frequency magnetic field pulse with the predetermined waveform.

17. The pulse sequence adjusting method according to claim 15,
wherein the prescan sequence includes a first prescan sequence, in which a magnetic resonance signal is measured by applying a readout gradient magnetic field of the same axis as the slice selection gradient magnetic field after applying the slice selection gradient magnetic field, and a second prescan sequence, in which the slice selection gradient magnetic field is different from that of the first prescan sequence, and
in the correction step, a correction value of the application start time of the slice selection gradient magnetic field in the imaging pulse sequence is calculated using the magnetic resonance signals acquired in the first and second prescan sequences.

18. The pulse sequence adjusting method according to claim 15,
wherein in the prescan step, a plurality of magnetic resonance signals is acquired by executing a plurality of prescan sequences with different application start time of the slice selection gradient magnetic field, and
in the correction step, a correction value of the application start time of the slice selection gradient magnetic field in the imaging pulse sequence is calculated using a plurality of magnetic resonance signals acquired using the plurality of prescan sequences with different application start time of the slice selection gradient magnetic field.

19. The pulse sequence adjusting method according to claim 15,
wherein in the correction step, a correction value calculated from a plurality of magnetic resonance signals measured with a plurality of gradient magnetic field delay values using a phantom is used.
